(19) United States
(12) Patent Application Publication
(10) Pub. No.: US 2004/0030203 A1
(43) Pub. Date: Feb. 12, 2004

(54) Method of stabilizing trichloroethane during production

(76) Inventors: Earl M. Gorton, Sulphur, LA (US); Ronald D. Olinger, Lake Charles, LA (US); Stephen D. Miller, Sulphur, LA (US)

Correspondence Address: Linda Pingitore, PPG Industries, Inc., One PPG Place, Pittsburgh, PA, 15272, US

(21) Appl. No.: 10/436,664

(22) Filed: May 13, 2003

Related U.S. Application Data
(60) Provisional application No. 60/396,460, filed on Jul. 16, 2002.

(51) Int. Cl. C07C 17/42

(52) U.S. Cl. 570/109

(57) ABSTRACT

Trichloroethane, e.g., 1,1,1-trichloroethane, is stabilized during processing at temperatures at which it is susceptible to thermal decomposition by conducting such processing in the presence of a stabilizing amount of a stable free radical stabilizer, e.g., a material having a 2,2,6,6-tetra(lower alkyl)-1-piperidinyloxy-yl free radical group such as 2,2,6,6-tetramethyl-4-hydroxy-1-piperidinyloxy.

### CROSS REFERENCE TO RELATED APPLICATIONS

This application claims priority to U.S. provisional patent application Serial No. 60/396,460 filed Jul. 16, 2002, which application is incorporated herein by reference in its entirety.

### DESCRIPTION OF THE INVENTION

The present invention relates to the stabilization of trichloroethanes. In particular, this invention relates to the stabilization of trichloroethanes, e.g., 1,1,1-trichloroethane and its isomer 1,1,2-trichloroethane, during high temperature processing, e.g., distillation. More particularly, this invention relates to stabilizing trichloroethanes during processing at temperatures at which the trichloroethanes are susceptible to thermal decomposition by performing such processing in the presence of a stabilizing amount of a stable free radical stabilizer, e.g., a material having a 2,2,6,6-tetra(lower alkyl)-1-piperidinyloxy-yl free radical group.

Other than in any operating examples, or where otherwise indicated, all numbers expressing quantities that are used in this specification and the accompanying claims are to be understood as modified in all instances by the term “about”.

### DETAILED DESCRIPTION OF THE INVENTION

1,1,1-Trichloroethane (viz., methyl chloroform) is commonly produced commercially by reacting 1,1-dichloroethane and molecular chlorine in the liquid phase and in the presence of free radical initiator. Similarly, the addition of molecular chlorine to chloroethene (viz., vinyl chloride) in the liquid phase to produce 1,1,2-trichloroethane is known. This latter reaction may proceed by an ionic path when a metal catalyst such as FeCl₃ is used, or by a radical path. See, for example, Kirk-Othmer Encyclopedia of Chemical Technology, third edition, volume 23, John Wiley & Sons, New York (1983), page 868, and Chemical Abstracts, volume 47, American Chemical Society, Columbus Ohio (1953), column 11218f, abstracting JP 26[1951]-6873. U.S. Pat. No. 6,150,573 discloses a method for the concurrent production of 1,1,1-trichloroethane and 1,1,2-trichloroethane by feeding molecular chlorine, chloroethene and 1,1-dichloroethane to a reaction vessel.

When raised to elevated temperatures during processing, chlorinated hydrocarbons, such as trichloroethanes, are prone to decomposition by thermal cracking, which produces undesirable side products. Such cracking can occur particularly during distillation of the chlorinated hydrocarbon product removed from the reaction vessel in a distillation zone containing a series of distillation columns. It would be desirable to suppress such decomposition by means of additives that suppress formation of decomposition products of the chlorinated hydrocarbons. In particular it would be desirable to reduce thermal cracking of 1,1,1-trichloroethane and/or 1,1,2-trichloroethane during high temperature processing of such trichloroethanes, which thermal cracking can lead to the formation of vinylidene chloride as a contaminant.

It has now been surprisingly discovered that the presence of a stable free radical suppresses thermal cracking of chlorinated hydrocarbons such as 1,1,1-trichloroethane and/or 1,1,2-trichloroethane during high temperature processing. An example of a type of stable free radical found to be particularly effective is characterized as having at least one 2,2,6,6-tetra(lower alkyl)piperidinyloxy-yl free radical group.

U.S. Pat. No. 6,040,488 discloses stabilizing vinylidene chloride against spontaneous polymerization with a free radical stabilizer having at least one 2,2,6,6-tetra(lower alkyl)-1-piperidinyloxy-yl free radical group. The stabilization described in the '488 patent relates to preventing the polymerization of vinylidene chloride in storage. It has no relevance to the problem of thermal cracking of chlorinated hydrocarbons during high temperature processing.

Any liquid phase reactor known to those skilled in the art for the production of chlorinated aliphatic hydrocarbons, e.g., trichloroethanes, can be used in conjunction with the process of the present invention. Preferably the reactor is of a type conventionally used for the production of 1,1,1-trichloroethane and/or 1.1.2-trichloroethane. It is equipped with inlets for the reactants and recycle stream from the purification zone, e.g., a distillation zone, an outlet for removal of gaseous hydrogen chloride, an outlet for removal of organic reaction product, and conventional means for regulating the temperature of the reaction mixture. Additional equipment such as an agitator, a vent condenser, pumps, heat exchangers, and the like may be employed, as desired.

In the production of 1,1,1-trichloroethane, 1,1-dichloroethane, molecular chlorine and free radical initiator are introduced to the reactor that contains a liquid reaction mixture. The reactants may be introduced as separate streams or two or more of the reactants may be combined prior to introduction. In many cases only a portion of the molecular chlorine introduced is available for the desired chlorination. This may be due to a variety of causes such as undesired side reactions and loss through the various outlets. It may be seen that the availability of chlorine atoms for the desired chlorination is a factor to be considered in choosing the relative proportions of molecular chlorine and organic feedstock to be used in conducting the reaction. Other factors to be considered include the degree to which the organic feedstock is to be chlorinated and the quantities and identities of other organic compounds, if any, which will be chlorinated. In general, sufficient molecular chlorine is introduced into the reactor to accomplish the desired degree of chlorination of the feedstock. Usually, but not necessarily, the mole ratio of molecular chlorine to 1,1-dichloroethane charged to the reactor is in the range of from 0.3:1 to 2.5:1. Often the ratio is in the range of from 0.4:1 to 2:1, e.g., from 0.5:1 to 2:1.

Free radical initiators that can be used in the production of 1,1,1-trichloroethane by the aforedescribed process are numerous and widely varied. See, for example, Kirk-Othmer Encyclopedia of Chemical Technology, third edition, volume 17, pages 1-90 (1982). In most cases, organic free radical initiators are used. One class of suitable organic free radical initiators comprises organic peroxygen-containing free radical initiators. This class of initiators can be divided into a large number of subclasses, some of which are as follows:

Aliphatic peroxides, which are exemplified by diethyl peroxide, di-tert-butyl peroxide [CAS 110-05-4], n-butyl 4,4-bis(tert-butylperoxy)valerate, 1,1-bis(tert-butylperoxy)-3,3,5-trimethylcyclohexane, bis-tert-butyl peroxides of diisopropylbenzene, dicumyl peroxide [CAS-80-43-3], 2,5-dimethyl-2,5-bis(tert-butylperoxy)hexane [CAS 78-63-7], and 2,5-dimethyl-2,5-bis(tert-butylperoxy)-3-hexyne [CAS 1068-27-5];

Hydroperoxides, which are exemplified by methyl hydroperoxide, tert-butyl hydroperoxide [CAS 75-91-2], cumyl hydroperoxide [CAS 80-15-9], 2,5-dimethyl-2,5-dihydroperoxyhexane [CAS 3025-88-5], p-menthanehydroperoxide [CAS 80-47-7], and diisopropylbenzene hydroperoxide [CAS 98-49-7];

Ketone peroxides, which are exemplified by methyl ethyl ketone peroxide, methyl isobutyl ketone peroxide, cyclohexanone peroxide, 2,4-pentanedione peroxide, the 1,2,4,5-tetraoxacyclohexanes, and the 1,2,4,5,7,8-hexaoxacyclononanes;

Aldehyde peroxides, which are exemplified by bis(1-hydroxyheptyl)peroxide;

Diperoxyketals, which are exemplified by 2,2-bis(tert-butylperoxy)butane [CAS 2167-23-9], ethyl 3,3-bis(tert-butylperoxy)butyrate [CAS 55794-20-2], 1,1-bis(tert -butylperoxy)cyclohexane [CAS 3006-86-8], and 1,1-bis(tert-butylperoxy)-3,3,5-trimethylcyclohexane [CAS 6731-36-8];

Diacyl peroxides, which are exemplified by diacetyl peroxide [CAS 110-22-5], dibenzoyl peroxide [CAS 94-36-0], dicaprylyl peroxide, bis(4-chlorobenzoyl)peroxide, didecanoyl peroxide, bis(2,4-dichlorobenzoyl)peroxide [CAS 133-14-2], diisobutyryl peroxide [CAS 3437-84-1], diisononanoyl peroxide, dilauroyl peroxide [CAS 105-74-8], dipelargonyl peroxide, dipropionyl peroxide, and bis(3-carboxylpropionyl)peroxide;

Peroxycarboxylic acids, which are exemplified by peroxyacetic acid;

Peroxyesters, which are exemplified by tert-butyl peroxyacetate [CAS 107-71-1], methyl peroxyacetate, tert-butyl peroxybenzoate [CAS 614-45-9], tert-butyl peroxy(2-ethylhexanonate) [CAS 3006-82-4], tert-butyl peroxyisobutyrate, tert-butyl peroxypivalate, 2,5-dimethyl-2,5-bis(benzoylperoxy)hexane [CAS 618-77-1], tert-butyl peroxy(2-ethylbutyrate), 2,5-dimethyl-2,5-bis(2-ethylhexanoylperoxy)hexane [CAS 13052-09-0], di-tert-butyl diperoxyazelate [CAS 16580-06-6], tert-amyl peroxy(2-ethylhexanoate) [CAS 686-31-7], di-tert-butyldiperoxyphthalate, 0,0-tert-butyl hydrogen monoperoxymaleate, dimethyl peroxyoxalate, di-tert-butyl diperoxyoxalate, and tert-butyl peroxyneodecanoate [CAS 748-41-4];

Peroxycarbonates, which are exemplified by tert-butylperoxy isopropyl carbonate; and

Peroxydicarbonates, which are exemplified by diisopropyl peroxydicarbonate [CAS 105-64-6], di-sec-butyl peroxydicarbonate, di-n-propyl peroxydicarbonate [CAS 16066-38-9], di(2-ethylhexyl)peroxydicarbonate, dicyclohexyl peroxydicarbonate [CAS 1561-49-5], and dicetyl peroxydicarbonate [CAS 26322-14-5].

Another class of suitable organic free radical initiators comprises the organic azo-nitrile initiators, of which there are many. Examples of suitable azo-nitrile initiators include 2,2′-azobis(2-methylpropanenitrile) [CAS 78-67-1], 2,2′-azobis(2-methylbutanenitrile) [CAS 13472-08-7], 2,2′-azobis(2,4-dimethylpentanenitrile) [CAS 4419-11-8], 2,2′-azobis(4-methoxy-2,4-dimethylpentanenitrile) [CAS 15545-97-8], 1,1′-azobis(cyclohexanecarbonitrile) [CAS 2094-98-6], 4.4′-azobis(4-cyanopentanoic acid) [CAS 2638-94-0], 2,2′-azobis(2-methylpentanenitrile), 2,2′-azobis(2,3-dimethylbutanenitrile), 2,2′-azobis(2-methylhexanenitrile), 2,2′-azobis(2,3-dimethylpentanenitrile), 2,2′-azobis(2,3,3-trimethylbutanenitrile), 2,2′-azobis(2,4,4-trimethylpentanenitrile), 2,2′-azobis(2-methyl-3-phenylpropanenitrile), 2,2′-azobis(2-cyclohexylpropanenitrile), 1,1′-azobis(cycloheptanecarbonitrile),1,1′-azobis(cyclooctanecarbonit rile), 1,1′-azobis(cyclodecanecarbonitrile), 2-(tert-butylazo)-4-methoxy-2,4-dimethylpentanenitrile [CAS 55912-17-9], 2-(tert-butylazo)-2,4-dimethylpentanenitrile [CAS 55912-18-0], 2-(tert-butylazo)-2-methylpropanenitrile [CAS 25149-46-6], 2-(tert-butylazo)-2-methylbutanenitrile [CAS 52235-20-8], 1-(tert -amylazo)cyclohexanecarbonitrile [CAS 55912-19-1], 1-(tert-butylazo)cyclohexanecarbonitrile [CAS 25149-47-7], and 2-[(1-chloro-1-phenylethyl)azo]-2-phenylpropanenitrile.

It is believed that many inorganic free radical initiators and metallic organic free radical initiators can also be used in the production of 1,1,1-trichloroethane. Examples of inorganic free radical initiators include sodium peroxide [CAS 1313-60-6], lithium peroxide [CAS 12031-80-0], potassium peroxide [CAS 17014-71-0], magnesium peroxide [CAS 14452-57-4], calcium peroxide [CAS 1305-79-9], strontium peroxide [CAS 1314-18-7], barium peroxide [CAS 1304-29-6], the sodium peroxyborates, sodium carbonate sesqui(peroxyhydrate) [CAS 15630-89-4], disodium peroxydicarbonate [CAS 3313-92-6], dipotassium peroxydicarbonate [CAS 589-97-9], monosodium peroxymonocarbonate [CAS 20745-24-8], monopotassium peroxymonocarbonate [CAS 19024-61-4], peroxymonophosphoric acid [CAS 13598-52-2], peroxydiphosphoric acid [CAS 13825-81-5], tetrapotassium peroxydiphosphate [CAS 15593-49-4], tetrasodium pyrophosphate bis[peroxyhydrate] [CAS 15039-07-3], peroxymonosulfuric acid [CAS 7722-86-3], oxone peroxymonosulfate [CAS 37222-66-5], peroxydisulfuric acid [CAS 13445-49-3], diammonium peroxydisulfate [CAS 7727-54-0], dipotassium peroxydisulfate [CAS 7727-21-1], disodium peroxydisulfate [CAS 7775-27-1], and zinc peroxide [CAS 1314-22-3].

Examples of metallic organic free radical initiators include, but are not limited to, diethyloxyaluminum tert-cumyl peroxide [CAS 34914-67-5], tri-tert-butyl perborate [CAS 22632-09-3], tert-butyl triethylgermanium peroxide [CAS 26452-74-4], dioxybis[triethylgermane] [CAS 58468-05-6], (tert-butyldioxy)triethylplumbane [CAS 18954-12-6], 00-tert-butyl dimethyl phosphorperoxoate [CAS 18963-64-9], tetrakis[tert-butyl]peroxysilicate [CAS 10196-46-0], dioxybis[trimethylsilane] [CAS 5796-98-5], (tert -butyldioxy)trimethylsilane [CAS 3965-63-7], dioxybis[triethylstannane] [CAS 4403-63-8], and (tert-butyldioxy)trimethylstannane [CAS 20121-56-6].

The amount of free radical initiator present in the liquid reaction mixture during the reaction can vary widely. The amount introduced depends upon many factors including, but not limited to: the identity and activity of the initiator; the composition of the organic feedstock; and the presence, identities, and concentrations, if any, of free radical poisons or inhibitors. In general, the free radical initiator is present in the liquid reaction mixture in at least an initiating amount. The minimum and maximum amounts are not limited by any theory, but by practical convenience. Since initiator deactivation is believed to proceed in at least some degree as the chlorination progresses and since it is difficult to ascertain how much active free radical initiator is present at any given instant, the relative proportions of free radical initiator and 1,1-dichloroethane are best expressed in terms of the weight ratios of these materials introduced to the reaction mixture, although it should be recognized that the amount of active free radical initiator present in the liquid phase reaction mixture is probably less at most times. If initiator deactivation is significant, the addition of free radical initiator may be made intermittently or continuously to remedy the problem. In most instances, the ratio of the weight of free radical initiator introduced to the reactor to the sum of the weight of the hydrocarbon reactants, e.g., 1,1-dichloroethane, or 1,1-dichloroethane and chloroethene, introduced into the reactor is in the range of from 50 to 5000 parts per million parts (ppm). Often the ratio is in the range of from 75 to 3000 ppm, e.g., from 100 to 1000 ppm.

The temperature at which the liquid phase chlorination is conducted can vary considerably. Usually, but not necessarily, the temperature is in the range of from 60° C. to 140° C., e.g., a temperature in the range of from 90° C. to 120° C.

The pressure at which the liquid phase chlorination is conducted can also vary widely. It may be subatmospheric, ambient atmospheric, or superatmospheric. In most cases it is at about ambient atmospheric pressure or somewhat higher. In many instances the pressure is in the range of from 0 to 1400 kilopascals, gauge. Often the pressure is in the range of from 100 to 1000 kilopascals, gauge, e.g., in the range of from 340 to 850 kilopascals, gauge.

Hydrogen chloride is removed from the reactor, usually as a gas, while the organic reaction product(s) can be removed from the reactor as a liquid or as a gas. In most instances the organic reaction product is removed as a liquid, but it may be vaporized and removed as a gas. In some circumstances, the organic reaction product is removed in both the liquid and gaseous state.

The organic reaction product removed from the reactor can be further processed as desired. In most cases, it is forwarded to a purification zone where the desired components are recovered as purified product compounds. The purification zone usually comprises a train of distillation columns. In a conventional purification zone, the organic reaction product removed from the reactor is forwarded to a first distillation column. An overhead stream comprising chiefly unreacted 1,1-dichloroethane is removed from or near the top of the first distillation column and is recycled to the reactor. A bottoms stream from the first distillation column comprising chiefly 1,1,1-trichloroethane, 1,1,2-trichloroethane, 1,2-dichloroethane and heavies is removed from or near the bottom of the first distillation column and is forwarded to a second distillation column. An overhead product stream comprising predominately 1,1,1-trichloroethane and some 1,2-dichloroethane is removed from or near the top of the second column. A bottoms stream comprising chiefly 1,1,2-trichloroethane and heavies is removed from or near the bottom of the second distillation column and is forwarded for further purification.

In the commercial chlorination process described above, the present invention can be practiced by providing a stabilizing amount of at least one stable free radical material in the chlorinated organic reaction liquid product that is subjected to high temperature processing. Because thermal cracking of the chlorinated hydrocarbon product, e.g., 1,1,1-trichloroethane, can occur in the distillation stages of the purification process, it is convenient to add the stable free radical material to the liquid product stream removed from the reactor prior to its being fed to the first (or recycle) distillation column. Alternatively, the stable free radical material can be added separately to the first distillation column. Stable free radical material charged to the first distillation column is removed with the bottoms liquid stream removed from that distillation column, and consequently forwarded and introduced into the second (or product) distillation column, thereby stabilizing the chlorinated hydrocarbon product processed in the second distillation column. In the event that there are further distillation columns in series with the first and second distillation columns, stable free radical material will be carried forward with the bottoms stream forwarded to the next successive distillation column.

The stable free radical stabilizer material can be characterized as having at least one 2,2,6,6-tetra(lower alkyl)piperidinyloxy-yl free radical group. The lower alkyl groups can be the same or they may be different, but usually they will be the same, and will comprise from 1 to 5, e.g., 1 to 4, carbon atoms. The lower alkyl group usually employed is methyl or ethyl, although lower alkyl groups having more than two carbon atoms, e.g., three or four carbon atoms, are contemplated. Typically, the lower alkyl group is methyl.

The 2,2,6,6-tetra(lower alkyl)piperidinyloxy-yl free radical group is usually the 2,2,6,6-tetra(lower alkyl)piperidinyloxy-4-yl free radical group, but the 2,2,6,6-tetra(lower alkyl)piperidinyloxy-3-yl free radical group may be used when desired. The 2,2,6,6-tetra(lower alkyl)piperidinyloxy-yl free radical group can be attached to hydrogen, hydroxyl, oxo, or to a parent compound as a substituent. In those embodiments in which the stable free radical is substituted onto a parent compound, the typical parent compound is a monocarboxylic acid or a dicarboxylic acid, in which case the stable free radical stabilizer material is an ester. The monocarboxylic acids can be aliphatic or aromatic. In one contemplated embodiment, the aliphatic monocarboxylic acid is saturated and contains from 1 to 18 carbon atoms. In other contemplated embodiments, the aliphatic monocarboxylic acid contains from 2 to 12 carbon atoms, e.g., from 3 to 8 carbon atoms. Of the aromatic monocarboxylic acids, benzoic acid is a particular embodiment. When dicarboxylic acids are used as the parent compound, the dicarboxylic acids can be saturated and contain from 2 to 13 carbon atoms. In one contemplated embodiment, the saturated dicarboxylic acid contains from 4 to 12 carbon atoms, e.g., from 8 to 12 carbon atoms. A particular contemplated embodiment of a saturated dicarboxylic acid is sebacic acid, which contains 10 carbon atoms. It should be understood that the stable free radical material of the present invention need not be associated with a parent compound, and in embodiments of the present invention, the stable free radical material itself is used.

The stable free radicals described herein and methods for their preparation are known to those skilled in the art. Non-limiting examples of suitable free radical materials that can be used in the present invention include:

2,2,6,6-tetramethyl-1-piperidinyloxy [CAS 2564-83-2];

2,2,6,6-tetramethyl-4-hydroxy-1-piperidinyloxy [CAS 2226-96-2] having the structure:

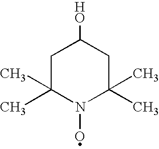

which material is also known as 4-hydroxy-TEMPO, and which is commercially available as a 5% active ingredient in an inert solvent mix from GE Betz as PETROFLO 20Y104;

2,2,6,6-tetramethyl-4-oxo-1-piperidinyloxy [CAS 2896-70-0];

2,2,6,6-tetramethyl-4-((methylsulfonyl)oxy)-1-piperidinyloxy [CAS 35203-66-8];

2,2,6,6-tetramethyl-1-piperidinyloxy-4-yl benzoate [CAS 3225-26-1]; and

bis(2,2,6,6-tetramethyl-1-piperidinyloxy-4-yl)sebacate [CAS 2516-92-9]

The amount of stable free radical stabilizer additive that is used in the present invention can vary and will depend on the amount of chlorinated product removed from the reactor and the amount charged to the distillation zone. In general, the amount of stable free radical stabilizer additive used can be characterized as a stabilizing amount. It is contemplated that at least one of the stable free radical stabilizers described above or their equivalents can be used. The amount of stable free radical additive added to the process stream will also depend upon the degree of stability desired and the effectiveness of the particular stable free radical employed. Minimal amounts may yield de minimis improvements. On the other hand diminishing returns are generally encountered when using amounts significantly larger than the amounts that provide an economically effective deterrent to thermal cracking, i.e., the formation of undesired levels of contaminating decomposition products. The upper and lower limits of practical effectiveness can be readily determined by the process operator by measuring the amounts of decomposition products in the product stream as the amount of stable free radical additive added to the system is varied. For example, the amount of vinylidene chloride in a 1,1,1-trichloroethane product stream can be measured by an on line gas chromatograph. It is contemplated that the amount of free radical stabilizer used will be sufficient to maintain the amount of vinylidene chloride in the trichloroethane product, e.g., 1,1,1- and 1,1,2-trichloroethane, at less than 500 ppm, e.g., not greater than 150 ppm.

It is contemplated that the stable free radical stabilizer will be present in amounts of from 0.1 to 10 parts per million parts (ppm) of the feed to the first (recycle) distillation column, e.g., from 0.4 to 5 ppm. In a particular contemplated production facility, it is contemplated that from 0.6 to 2.6 ppm, e.g., 0.6 to 1.5 ppm, of stable free radical stabilizer is added to the first distillation column. The amount of stable free radical stabilizer used can vary in amounts ranging between any of these upper and lower values, inclusive of the recited values.

The concentration of stable free radical stabilizer in the bottoms stream removed from the first (recycle) distillation column and forwarded to the second (product) distillation column is higher than the concentration in the first distillation column because the volume of feed to the second distillation column is lower than the feed to the first distillation column, and the amount of stable free radical additive in the bottoms stream removed from the first distillation column is substantially the same as that charged to the first distillation column.

It is contemplated that the stable free radical stabilizer will be present in the feed to the second distillation column in amounts of from 0.2 to 20 parts per million parts of feed (ppm) to the second distillation column, e.g., from 0.8 to 10 ppm. In a particular contemplated production facility, it is contemplated that from 1.2 to 2.5 ppm of stable free radical stabilizer is present in the second distillation column. As indicated above, the concentration of stable free radical stabilizer in the second distillation column can vary between any of these upper and lower values, inclusive of the recited values. Successive distillation columns following the second distillation column (where used) will contain higher amounts of stable free radical stabilizer than the amount found in the second distillation column.

Stabilization can be shown by determining the amount of vinylidene chloride in the trichloroethane product with and without use of the stable free radical stabilizer additive. Reduced levels of vinylidene chloride in the trichloroethane product show that thermal cracking of trichloroethane has been retarded.

The present invention is further described in the following example, which is to be considered as illustrative, rather than limiting, of the invention, and wherein all parts are parts by weight and all percentages are percentages by weight unless specified otherwise.

### EXAMPLE

1,1,1-Trichloroethane was produced in the liquid phase in a conventional reactor by the reaction of 1,1-dichloroethane and chlorine in the presence of a free radical initiator. A crude product stream was removed from the reactor and forwarded to a first (recycle) distillation column. A portion of the feed to the first distillation column was removed as an overhead stream and recycled to the reactor, while a second portion was removed as bottoms from this distillation column. The bottoms stream from the first distillation column was forwarded (as feed) to a second (product) distillation column. A portion of the feed to the second distillation column was removed overhead as 1,1,1-trichloroethane product, and a further portion of the feed was removed as a bottoms stream for further processing. Both distillation columns were operated under reflux conditions.

Analysis of the product distillation column reflux for vinylidene chloride (VDC) by gas chromatography before addition of a free radical stabilizer found a concentration of 212 ppm of VDC. 4-hydroxy-TEMPO (as PETROFLO 20Y104) was added to the feed to the first distillation column in amounts that provided a concentration of 4-hydroxy-TEMPO in the feed of approximately 2.6 ppm. After 3½ hours of operation, the product distillation column reflux was analyzed again for VDC and found to contain 61 ppm. This shows a reduction of VDC of almost 3.5 times in the product reflux, which is dramatic evidence of the reduction of thermal cracking of trichloroethane by use of a free radical stabilizer.

Although the present invention has been described with reference to specific details of certain embodiments thereof, it is not intended that such details should be regarded as limitations upon the scope of the invention except insofar as they are included in the accompanying claims.

What is claimed is:

1. A method for stabilizing trichloroethane during processing at temperatures at which trichloroethane is susceptible to thermal decomposition, comprising conducting said processing in the presence of a stabilizing amount of a stable free radical stabilizer.

2. The method of claim 1 wherein the stable free radical stabilizer is a material having a 2,2,6,6-tetra(lower alkyl)-1-piperidinyloxy-yl free radical group.

3. The method of claim 2 wherein the 2,2,6,6-tetra(lower alkyl)-1-piperidinyloxy-yl free radical is a material having a 2,2,6,6, tetramethyl-1-piperidinyloxy-yl free radical group.

4. The method of claim 1 wherein the stable free radical stabilizer is a material having a 2,2,6,6-tetra(lower alkyl)-1-piperidinyloxy-4-yl free radical group.

5. The method of claim 4 wherein the stable free radical stabilizer is a material having a free radical group selected from the 2,2,6,6-tetramethyl-4-hydroxy-1-piperidinyloxy, the 2,2,6,6-tetramethyl-4-oxo-1-piperidinyloxy, or the 2,2,6,6-tetramethyl-4-[(methylsulfonyl)oxy]-1-piperidinyloxy free radical group.

6. The method of claim 4 wherein the stable free radical stabilizer is a material having a 2,2,6,6-tetramethyl-1-piperidinyloxy-4-yl benzoate free radical group.

7. The method of claim 4 wherein the stable free radical stabilizer is a bis(2,2,6,6-tetramethyl-1-piperidinyloxy-4-yl)ester of a saturated dicarboxylic acid.

8. The method of claim 7 wherein the saturated dicarboxylic acid contains from 2 to 13 carbon atoms.

9. The method of claim 8 wherein the stable free radical stabilizer is bis(2,2,6,6-tetramethyl-1-piperidinyloxy-4-yl)sebacate.

10. The method of claim 1 wherein the stable free radical stabilizer group is present in amounts of from 0.1 to 10 parts per million parts of the composition comprising trichloroethane that is processed at temperatures at which the trichloroethane is susceptible to thermal decomposition.

11. The method of claim 10 wherein the stable free radical stabilizer group is present in amounts of from 0.6 to 2.6 parts per million parts of the composition comprising trichloroethane that is processed.

12. The method of claim 10 wherein the trichloroethane is selected from 1,1,1-trichloroethane, 1,1,2-trichloroethane and mixtures of 1,1,1-trichloroethane and 1,1,2-trichloroethane.

13. The method of claim 11 wherein the processing performed is distillation.

14. A process of distilling a composition comprising trichloroethane selected from 1,1,1-trichloroethane, 1,1,2-trichloroethane and mixtures of 1,1,1-trichloroethane and 1,1,2-trichloroethane, comprising performing the distillation in the presence of a stabilizing amount of a stable free radical stabilizer having a 2,2,6,6-tetra(lower alkyl)-1-piperidinyloxy-yl free radical group.

15. The process of claim 14 wherein the 2,2,6,6-tetra(lower alkyl)-1-piperidinyloxy-yl free radical group is a 2,2,6,6, tetramethyl-1-piperidinyloxy-yl free radical group.

16. The process of claim 14 wherein the stable free radical stabilizer is a material having a 2,2,6,6-tetra(lower alkyl)-1-piperidinyloxy-4-yl free radical group.

17. The process of claim 16 wherein the stable free radical stabilizer is a material having a free radical group selected from the 2,2,6,6-tetramethyl-4-hydroxy-1-piperidinyloxy, the 2,2,6,6-tetramethyl-4-oxo-1-piperidinyloxy, or the 2,2,6,6-tetramethyl-4-[(methylsulfonyl)oxy]-1-piperidinyloxy free radical group.

18. The process of claim 14 wherein the stable free radical stabilizer is present in amounts of from 0.4 to 5 parts per million parts of the composition comprising trichloroethane that is distilled.

19. The process of claim 15 wherein the stable free radical stabilizer is present in amounts of from 0.6 to 2.6 parts per million parts of the composition comprising trichloroethane that is distilled.

20. The process of claim 14 wherein the stable free radical stabilizer group is 2,2,6,6-tetramethyl-4-hydroxy-1-piperidinyloxy, which is present is amounts of from 0.6 to 1.5 parts per million parts of the composition comprising trichloroethane that is distilled.
